(19) United States
(12) Patent Application Publication
(10) Pub. No.: US 2007/0099951 A1
(43) Pub. Date: May 3, 2007

(54) 4-oxo-1-(3-substituted phenyl)-1,4-dihydro-1,8-naphthyridine-3-carboxamide phosphodiesterase-4 inhibitor and a method of preparing same

(76) Inventors: Daniel Dube, Saint-Lazare (CA); Michel Gallant, Kirkland (CA); Patrick Lacombe, Montreal (CA)

Correspondence Address: MERCK AND CO., INC, P O BOX 2000, RAHWAY, NJ, 07065-0907, US

(21) Appl. No.: 11/582,697

(22) Filed: Oct. 18, 2006

Related U.S. Application Data
(60) Provisional application No. 60/730,621, filed on Oct. 27, 2005.

(51) Int. Cl. A61K 31/4745; A61K 31/415; A61K 31/401; A61K 31/366; A61K 31/22; C07D 471/02

(52) U.S. Cl. 514/300; 514/406; 514/423; 514/460; 514/548; 546/123

(57) ABSTRACT

The invention is directed to a compound of the structural formula (22) 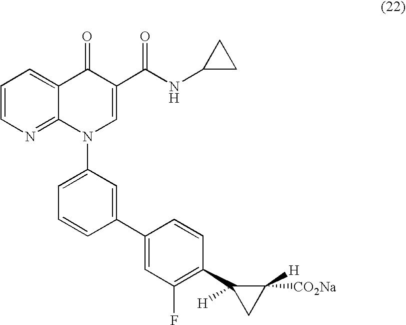 crystal form of structural formulae (21) and its free acid, pharmaceutical compositions comprising these compounds and methods of preparing and using these compounds.

### BACKGROUND OF THE INVENTION

1. Field of the Invention

The invention is directed to a compound of the structural formula (22) 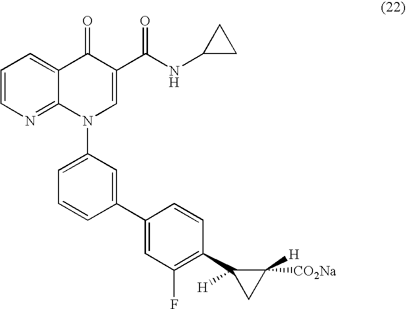
crystal forms of structural formulae (22) and its free acid, pharmaceutical compositions comprising these compounds and methods of preparing and using these compounds.

2. Related Background

Hormones are compounds that variously affect cellular activity. In many respects, hormones act as messengers to trigger specific cellular responses and activities. Many effects produced by hormones, however, are not caused by the singular effect of just the hormone. Instead, the hormone first binds to a receptor, thereby triggering the release of a second compound that goes on to affect the cellular activity. In this scenario, the hormone is known as the first messenger while the second compound is called the second messenger. Cyclic adenosine monophosphate (adenosine 3′, 5′-cyclic monophosphate, “cAMP” or “cyclic AMP”) is known as a second messenger for hormones including epinephrine, glucagon, calcitonin, corticotrophin, lipotropin, luteinizing hormone, norepinephrine, parathyroid hormone, thyroid-stimulating hormone, and vasopressin. Thus, cAMP mediates cellular responses to hormones. Cyclic AMP also mediates cellular responses to various neurotransmitters.

Phosphodiesterases (“PDE”) are a family of enzymes that metabolize 3′, 5′ cyclic nucleotides to 5′ nucleoside monophosphates, thereby terminating cAMP second messenger activity. A particular phosphodiesterase, phosphodiesterase-4 (“PDE4”, also known as “PDE-IV”), which is a high affinity, cAMP specific, type IV PDE, has generated interest as potential targets for the development of novel anti-asthmatic and anti-inflammatory compounds. PDE4 is known to exist as at lease four isoenzymes, each of which is encoded by a distinct gene. Each of the four known PDE4 gene products is believed to play varying roles in allergic and/or inflammatory responses. Thus, it is believed that inhibition of PDE4, particularly the specific PDE4 isoforms that produce detrimental responses, can beneficially affect allergy and inflammation symptoms. It would be desirable to provide novel compounds and compositions that inhibit PDE4 activity.

A major concern with the use of PDE4 inhibitors is the side effect of emesis which has been observed for several candidate compounds as described in C.Burnouf et al., (“Burnouf”), Ann. Rep. In Med Chem., 33:91-109(1998). B. Hughes et al., Br. J. Pharmacol., 118:1183-1191(1996); M. J. Perry et al., Cell Biochem. Biophys., 29:113-132(1998); S. B. Christensen et al., J. Med. Chem., 41:821-835(1998); and Burnouf describe the wide variation of the severity of the undesirable side effects exhibited by various compounds. As described in M. D. Houslay et al., Adv. In Pharmacol, 44:225-342(1998) and D. Spina et al., Adv. In Pharmacol, 44:33-89(1998), there is great interest and research of therapeutic PDE4 inhibitors.

International Patent Publication W09422852 describes quinolines as PDE4 inhibitors. International Patent Publication W09907704 describes 1-aryl-1,8-naphthylidin-4-one derivatives as PDE4 inhibitors.

WO2004/048374, published Jun. 10, 2004, discloses the compound of Formula (21) and a process for making same.

WO2004/048377, published Jun. 10, 2004 and U.S. Pat. No. 6,909,002, issued Jun. 21, 2005 discloses processes useful for making naphthyridene PDE4 inhibitors.

### SUMMARY OF THE INVENTION

The invention is directed to a compound of the structural formula (22) 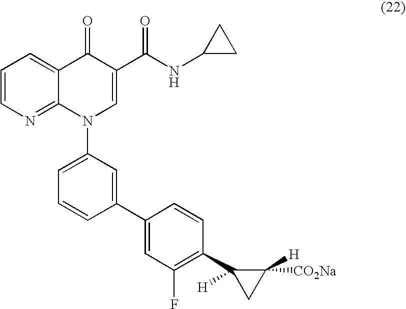
crystal forms of structural formulae (22) and its free acid, pharmaceutical compositions comprising these compounds and methods of preparing and using these compounds.

### BRIEF DESCRIPTION OF THE FIGURES

FIG. 1 is a characteristic X-ray diffraction pattern of the crystalline sodium salt of structural formula (22).

FIG. 2 is a carbon-13 cross-polarization magic-angle spinning (CPMAS) nuclear magnetic resonance (NMR) spectrum of the crystalline sodium salt of structural formula (22).

FIG. 3 is a fluorine-19 magic-angle spinning (MAS) nuclear magnetic resonance (NMR) spectrum of the crystalline sodium salt of structural formula (22).

FIG. 4 is a typical Raman spectrum of the crystalline sodium salt of formula (22).

FIG. 5 is a characteristic X-ray diffraction pattern of the crystalline free acid of structural formula (21).

FIG. 6 is a carbon-13 cross-polarization magic-angle spinning (CPMAS) nuclear magnetic resonance (NMR) spectrum of the crystalline free acid of structural formula (21).

FIG. 7 is a fluorine-19 magic-angle spinning (MAS) nuclear magnetic resonance (NMR) spectrum of the crystalline free acid of structural formula (21).

FIG. 8 is a typical differential scanning calorimetry (DSC) curve of the free acid of structural formula (21).

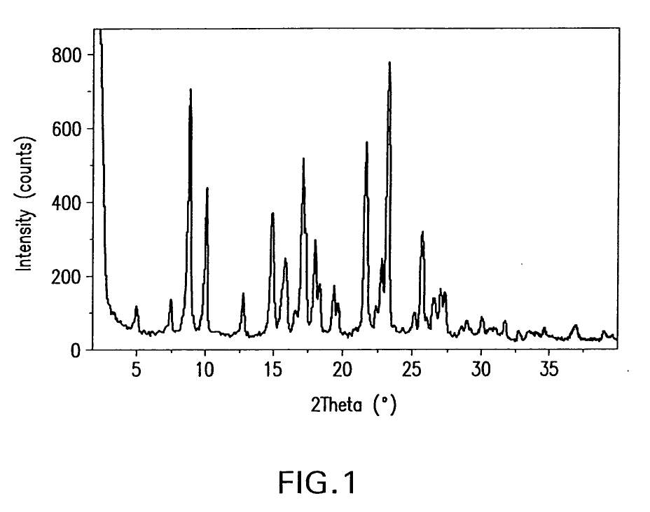

Major peaks from FIG. 1 are as shown below (wavelength CuKalpha):

| 2 theta | d-spacing |
|---|---|
| 8.8 | 10.05 |
| 17.2 | 5.16 |
| 10.1 | 8.76 |
| 23.2 | 3.83 |
| 4.90 | 4.11 |
| 14.9 | 5.95 |
| 5.0 | 17.67 |
| 15.9 | 5.57 |
| 18.1 | 4.90 |

Table: Major peaks from FIG. 5 are as shown below (wavelength Cu Kalpha).

| 2 theta | d-spacing |
|---|---|
| 5.4 | 16.37 |
| 15.3 | 5.79 |
| 18.3 | 4.85 |
| 6.4 | 13.81 |
| 10.4 | 8.51 |
| 6.1 | 14.49 |
| 7.0 | 12.63 |
| 8.2 | 10.78 |
| 9.6 | 9.21 |

### DETAILED DESCRIPTION OF THE INVENTION

In one aspect, the invention is directed to a compound of the Formula (22)

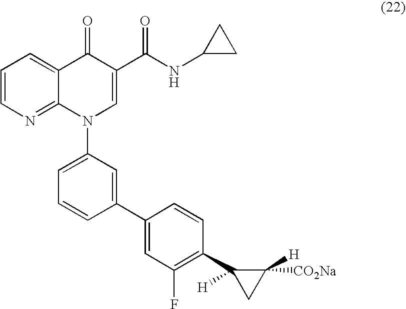

In another aspect, there are pharmaceutical compositions comprising a compound of structural formula (22) and a pharmaceutically acceptable carrier.

Within this aspect, there is a genus of pharmaceutical composition further comprising a Leukotriene receptor antagonist, a Leukotriene biosynthesis inhibitor, an M2/M3 antagonist, a corticosteroid, an H1 receptor antagonist or a beta 2 adrenoceptor agonist.

Within this aspect, there is another genus of pharmaceutical composition further comprising a COX-2 selective inhibitor, a statin, or an NSAID.

In another aspect, the invention is directed to a method of treatment or prevention of asthma, chronic bronchitis, chronic obstructive pulmonary disease (COPD), eosinophilic granuloma, psoriasis and other benign or malignant proliferative skin diseases, endotoxic shock (and associated conditions such as laminitis and colic in horses), septic shock, ulcerative colitis, Crohn's disease, reperfusion injury of the myocardium and brain, inflammatory arthritis, osteoporosis, chronic glomerulonephritis, atopic dermatitis, urticaria, adult respiratory distress syndrome, infant respiratory distress syndrome, chronic obstructive pulmonary disease in animals, diabetes insipidus, allergic rhinitis, allergic conjunctivitis, vernal conjunctivitis, arterial restenosis, atherosclerosis, neurogenic inflammation, pain, cough, rheumatoid arthritis, ankylosing spondylitis, transplant rejection and graft versus host disease, hypersecretion of gastric acid, bacterial, fungal or viral induced sepsis or septic shock, inflammation and cytokine-mediated chronic tissue degeneration, osteoarthritis, cancer, cachexia, muscle wasting, depression, memory impairment, monopolar depression, acute and chronic neurodegenerative disorders with inflammatory components, Parkinson disease, Alzheimer's disease, spinal cord trauma, head injury, multiple sclerosis, tumour growth and cancerous invasion of normal tissues comprising the step of administering a therapeutically effective amount, or a prophylactically effective amount, of the compound of structural formula (22).

In another aspect, the invention is directed to a method of enhancing cognition in a subject comprising administering a safe cognition enhancing amount of compound of structural formula (22).

In another aspect, the invention is directed to a crystalline form of the compound of structural formula (22).

In another aspect, the invention is directed to a crystalline form of the compound of structural formula (21)

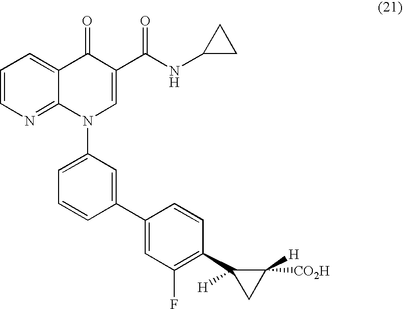

In another aspect, there are pharmaceutical compositions comprising crystalline compound of structural formula (21) or (22) and a pharmaceutically acceptable carrier.

Within this aspect, there is a genus of pharmaceutical composition further comprising a Leukotriene receptor antagonist, a Leukotriene biosynthesis inhibitor, an M2/M3 antagonist, a corticosteroid, an Hi receptor antagonist or a beta 2 adrenoceptor agonist.

Within this aspect, there is another genus of pharmaceutical composition further comprising a COX-2 selective inhibitor, a statin, or an NSAID.

In another aspect, the invention is directed to a method of treatment or prevention of asthma, chronic bronchitis, chronic obstructive pulmonary disease (COPD), eosinophilic granuloma, psoriasis and other benign or malignant proliferative skin diseases, endotoxic shock (and associated conditions such as laminitis and colic in horses), septic shock, ulcerative colitis, Crohn's disease, reperfusion injury of the myocardium and brain, inflammatory arthritis, osteoporosis, chronic glomerulonephritis, atopic dermatitis, urticaria, adult respiratory distress syndrome, infant respiratory distress syndrome, chronic obstructive pulmonary disease in animals, diabetes insipidus, allergic rhinitis, allergic conjunctivitis, vernal conjunctivitis, arterial restenosis, atherosclerosis, neurogenic inflammation, pain, cough, rheumatoid arthritis, ankylosing spondylitis, transplant rejection and graft versus host disease, hypersecretion of gastric acid, bacterial, fungal or viral induced sepsis or septic shock, inflammation and cytokine-mediated chronic tissue degeneration, osteoarthritis, cancer, cachexia, muscle wasting, depression, memory impairment, monopolar depression, acute and chronic neurodegenerative disorders with inflammatory components, Parkinson disease, Alzheimer's disease, spinal cord trauma, head injury, multiple sclerosis, tumour growth and cancerous invasion of normal tissues comprising the step of administering a therapeutically effective amount, or a prophylactically effective amount, of the crystalline compound of structural formula (21) or (22).

In another aspect, the invention is directed to a method of enhancing cognition in a subject comprising administering a safe cognition enhancing amount of crystalline compound of structural formula (21) or (22).

In another aspect the invention is directed to a composition comprising a crystalline salt of the compound of structural formula (22) and a detectable amount of a free acid of the structural formula (21) wherein said free acid is optionally crystalline.

Within this aspect there is a genus comprising about 5% to about 100% by weight of said optionally crystalline free acid.

Within this aspect there is a genus comprising about 10% to about 100% by weight of said optionally crystalline free acid.

Within this aspect there is a genus comprising about 25% to about 100% by weight of said optionally crystalline free acid.

Within this aspect there is a genus comprising about 50% to about 100% by weight of said optionally crystalline free acid.

Within this aspect there is a genus comprising about 75% to about 100% by weight of said optionally crystalline free acid.

Within this aspect there is a genus comprising substantially all of said optionally crystalline free acid.

In one aspect the invention is directed to a method of making a compounds of Formulae (20), (21) and (22): 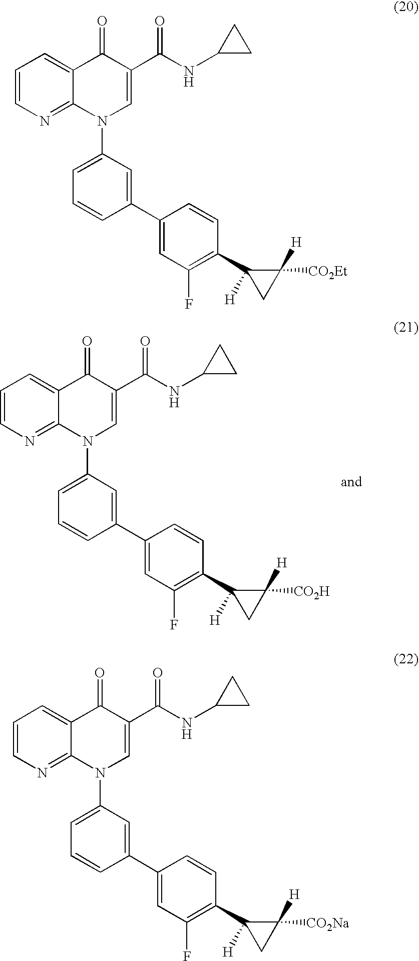
Comprising:
Step (a) reacting a compound of the Formula (5) 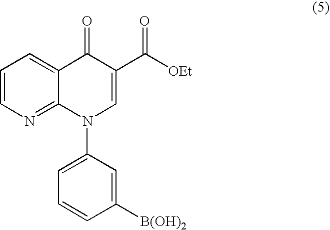
   in a first solvent with pinacol 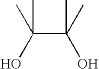
   to provide an ester of the Formula (15) 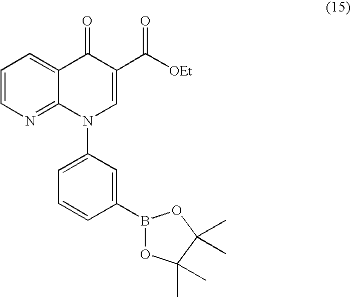
Step (b) reacting an ester of the Formula (15) in an aprotic solvent with Lewis acid and cyclopropylamine 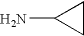
   followed by acidic aqueous work up to provide a compound of Formula (16) 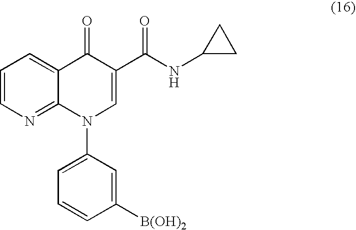
Step (c) reacting a compound of Formula (16) with an aryl bromide of Formula (3) 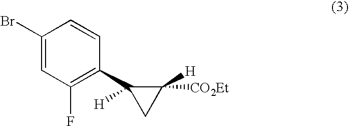
   in a suspension of a palladium catalyst and a phosphine ligand in a third solvent followed by addition of aqueous buffer to provide a compound of Formula (20) 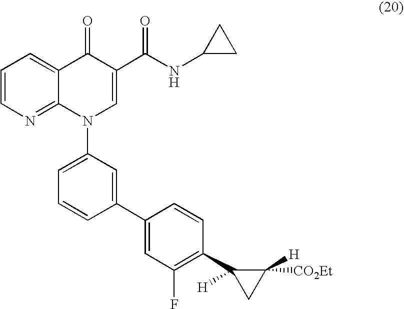
Step (d) reacting a compound of the Formula (20)
   With a strong base in an C₁₋₆alkanol solvent to provide a compound of Formula (21) 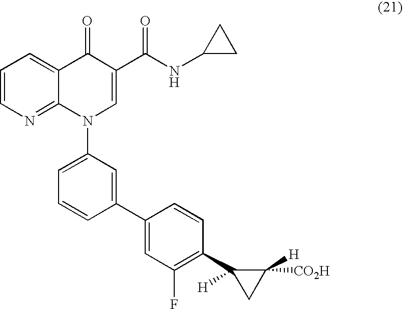
Step (e) reacting a compound of Formula (21)

with a sodium base in a solvent comprising water and an C₁₋₆alkanol solvent to provide a compound of the Formula (22)

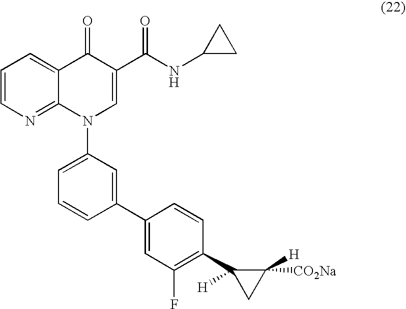

Regarding Step (a), the molar ratio of the compound of Formula (5) to pinacol is approximately 0.5:1 to 2:1 and is typically approximately 1:1, with a modest excess of the pinacol. For purposes of this specification, the first solvent is defined as any non-reactive solvent capable of removing water by azeotropic distillation. The first solvent includes solvents such as toluene and xylene. Reaction Step (a) may be conveniently carried out at a temperature range of 60 to 120° C.; typically 80 to 110° C. and is allowed to proceed until substantially complete in 1 to 6 hours; typically 2 to 4 hours.

Regarding Step (b), the molar ratio of the compound of Formula (15) to Lewis acid is approximately 0.5:1 to 2:1 and is typically approximately 1:1 with an excess of the ester. The molar ratio of the compound of Formula (15) to cyclopropylamine is approximately 0.8:1 to 1:6 and is typically approximately 1:3 to 1:5. For purposes of this specification, the aprotic solvent is defined to include Dimethyl acetamide (DMAc) and Dimethyl formamide (DMF). For purposes of this reaction step, the Lewis acid is defined to include MgCl₂ and ZnCl₂. Reaction Step (b) may be conveniently carried out at a temperature range of 0 to 60° C.; typically 15 to 50° C. and is allowed to proceed until substantially complete in 1 to 6 hours; typically 2 to 4 hours.

Regarding Step (c), the molar ratio of the compound of Formula (16) to the compound of Formula (3) is approximately 0.5:1 to 2.0:1 and is typically approximately 1:1. The molar ratio of the palladium catalyst to compound of Formula 16 is approximately 0.001:1 to 0.1:1 and is typically 0.02:1 to 0.05:1. The molar ratio of aqueous buffer to compound of Formula (16) is 2:1 or greater. The aqueous buffer includes buffers such as sodium carbonate, potassium carbonate, sodium phosphate, and potassium pposphate. The molar ratio of the phosphine ligand to compound of Formula 16 is approximately 0.05:1 to 0.5:1 and is typically 0.1:1 to 0.3:1 For purposes of this specification, the third solvent is defined to include Dimethyl formamide, propanol, including n-propanol and mixtures of these solvents. The phosphine ligand is defined to include P(Cl₆alkyl)₃, such as P(t-butyl)₃, P(Cy)₃, and P(t-butyl)₂(biphenyl) or P(aryl)3, such as (phenyl)3. For purposes of this specification, the palladium catalyst includes Fu's catalyst (i.e. P(t-butyl)₃-Pd-P(t-butyl)₃), [PdCl(allyl)]₂, Pd₂ (dba)₃, and [P(t-butyl)₃PdBr]₂ (Johnson-Matthey catalyst). Reaction Step (c) may be conveniently carried out at a temperature range of 0 to 100° C.; typically 20 to 85° C. and is allowed to proceed until substantially complete.

Regarding Step (d), the molar ratio of the compound of Formula (20) to NaS₂O₅ is approximately 1:0.05 to 1:0.2 and is typically approximately 1:0.1. The molar ratio of compound of Formula (20) to strong base is approximately 1:2 to 1:4 and is typically 1:3 or greater. The strong bas included sodium hydroxide. For purposes of this specification, the Cl-6alkanol solvent is defined to include methanol, ethanol, i-propanol and n-propanol. Reaction Step (d) is allowed to proceed until substantially complete in 0.5 to 4 hours; typically 1 to 3 hours.

Regarding Step (e), the molar ratio of the compound of Formula (21) to sodium base is approximately 0.5:1 to 2.0:1.05 and is typically approximately 1:1 or an excess of sodium base. For purposes of this specification, the C1-6alkanol solvent is defined as for step (d). For purposes of this specification, the sodium base is defined to include sodium hydroxide and sodium alkoxide such as sodium methoxide. Reaction Step (e) may be conveniently carried out at a temperature range of 0 to 100° C.; typically 20 to 80° C. and is allowed to proceed until substantially complete.

Within this aspect there is a genus wherein
the aprotic solvent is dimethylacetamide or dimethylformamide;
the Lewis acid is MgCl₂ or ZnCl₂;
the palladium catalyst is P(t-butyl)₃-Pd-P(t-butyl)₃), [PdCl(allyl)]₂, Pd₂ (dba)₃ or [P(t-butyl)₃PdBr]₂;
the phosphine ligand is P(t-butyl)₃, P(Cy)₃, l) or P(phenyl)₃;
the third solvent is dimethylformamide or propanol or a mixture thereof;
the strong base is sodium hydroxide;
the sodium base is sodium hydroxide or sodium alkoxide.
the C₁-6alkanol solvent is methanol, ethanol, i-propanol, or n-propanol; and
the aqueous buffer is a sodium carbonate.

In another aspect, the invention encompasses a process of making an intermediate compound of the Formula (3) 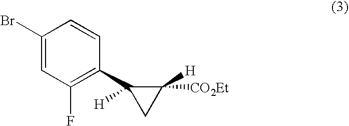
comprising
Step (f) reacting in absence of oxygen a copper(I) trifluoromethanesulfonate benezene complex in MTEB (methyl t-butyl ether) with bisoxazoline ligand of Formula (10) 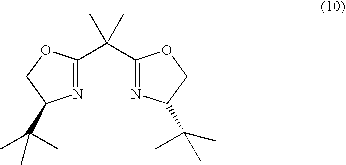
   to provide a copper(I) catalyst believed to have the Formula (10-Cu) 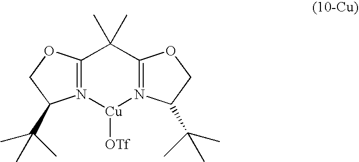
Step (g) reacting a vinylbenzene of Formula (2) 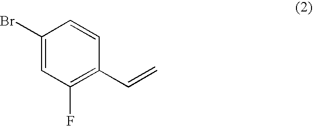
   with ethyl diazoacetate in MTEB in the presence of the copper (I) catalyst of Formula (10-Cu) to produce a compound of the Formula (3)

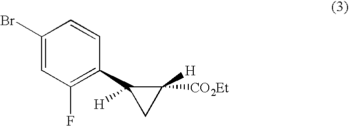

Regarding Step (f), the molar ratio of the ligand of Formula (10) to the copper(I) trifluoromethanesulfonate benezene complex is approximately 0.5:1 to 2.0:1 and is typically approximately 1:1 to 1.5:1. For purposes of this specification, the solvent is defined to include Methyl t-butyl ether, THF, hexanes, heptane and toluene. Reaction Step (f) may be conveniently carried out at a temperature range of 0 to 50° C.; typically 10 to 30° C. and is allowed to proceed until substantially complete in 0.5 to 2 hours.

Regarding Step (g), the molar ratio of the vinylbenzene of Formula (2) to ethyl diazoacetate is approximately 0.3: 1 to 2.0:1 and is typically approximately 1:2. For purposes of this specification, the solvent is defined to include Methyl t-butyl ether, THF, hexanes, heptane and toluene. Reaction Step (g) is allowed to proceed until substantially complete.

In another aspect, the invention encompasses a process of making an intermediate compound of the Formula (2) 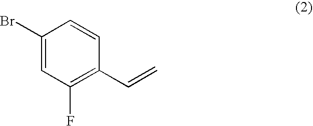
Comprising
Step (h) reacting a compound of the Formula (1) 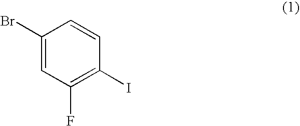
   with vinyl magnesium chloride of the Formula 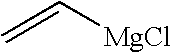
   and ZnCl₂ in a hydrocarbon solvent in the presence of a phosphine ligand and a palladium catalyst to provide a compound of the Formula (2)

Regarding Step (h), the molar ratio of the compound of Formula (1) to vinyl magnesium chloride is approximately 0.3:1 to 3:1 and is typically approximately 1:2. The molar ratio of the compound of Formula (1) to ZnCl₂ is approximately 1:1. For purposes of this specification, the hydrocarbon solvent is defined to include THF, pentanes, hexanes, hexane and toluene. For purposes of this specification the phosphine ligand is defined to include P(C₁₋₆alkyl)₃, such as P(t-butyl)₃, P(Cy)₃, P(t-butyl)₂(biphenyl) and P(aryl)₃, such as P(phenyl)₃. For purposes of this specification, the palladium catalyst includes Fu's catalyst (i.e. P(t-butyl)₃-Pd-P(t-butyl)₃), [PdCl(allyl)]₂, Pd₂ (dba)₃, and [P(t-butyl)₃PdBr]₂ (Johnson-Matthey catalyst). Reaction Step (h) is allowed to proceed until substantially complete in 1 to 10 hours; typically 2 to 6hours.

Within this aspect there is a genus wherein the hydrocarbon solvent is pentane or hexane; the phosphine ligand is P(t-butyl)₃, P(Cy)₃, P(t-butyl)₂(biphenyl) or P(phenyl)₃. the palladium catalyst is P(t-butyl)₃-Pd-P(t-butyl)₃), [PdCl(allyl)]₂, Pd₂ (dba)₃ or [P(t-butyl)₃PdBr]₂.

In a further aspect is a process for a method of increasing the purity of a compound of Formula (3) 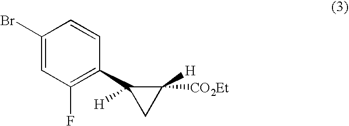
by removing its cis counterpart, a compound of Formula (3-cis)
and Compounds of Formula (11) and (12) 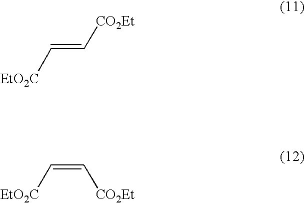
Comprising
Step (i) reacting said preparation with a reducing agent such as sodium borohydride in C₁₋₆alkanol to reduce Compounds of formula (11) and (12) to a compound of Formula ( I1 a) 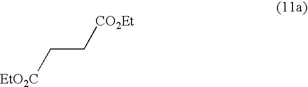
   and removing the compound of Formula (11a) and 3-cis by Step (j) hydrolyzing the products of Step (i) with LiOH to convert the Compound of Formula (3) to a Compound of Formula (13) or its Li salt and to convert the compound of formula (11a) to its diacid or lithium salt; 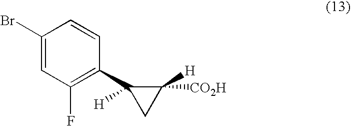
Step (k) removing cis-3 by extraction with an organic solvent such as MTBE, heptane, and/or their mixtures.
Step (l) purifying the compound of formula 13 by crystallization from a suitable crystallizing solvent such as methanol, water or mixtures thereof;
Step (m) reacting of the compound of formula 13 with ethanol and thionyl chloride to form compound of formula (3).

Compounds of Formula (21) and (22) are useful Inhibitors of phosphodiesterase-4 useful in the treatment in mammals of, for example, asthma, chronic bronchitis, chronic obstructive pulmonary disease (COPD), eosinophilic granuloma, psoriasis and other benign or malignant proliferative skin diseases, endotoxic shock (and associated conditions such as laminitis and colic in horses), septic shock, ulcerative colitis, Crohn's disease, reperfusion injury of the myocardium and brain, inflammatory arthritis, osteoporosis, chronic glomerulonephritis, atopic dermatitis, urticaria, adult respiratory distress syndrome, infant respiratory distress syndrome, chronic obstructive pulmonary disease in animals, diabetes insipidus, allergic rhinitis, allergic conjunctivitis, vernal conjunctivitis, arterial restenosis, atherosclerosis, neurogenic inflammation, pain, cough, rheumatoid arthritis, ankylosing spondylitis, transplant rejection and graft versus host disease, hypersecretion of gastric acid, bacterial, fungal or viral induced sepsis or septic shock, inflammation and cytokine-mediated chronic tissue degeneration, osteoarthritis, cancer, cachexia, muscle wasting, depression, memory impairment, monopolar depression, acute and chronic neurodegenerative disorders with inflammatory components, Parkinson disease, Alzheimer's disease, spinal cord trauma, head injury, multiple sclerosis, tumour growth and cancerous invasion of normal tissues.

The pharmaceutical compositions of the present invention comprise a compound represented by Formula (21) or (22) as an active ingredient, a pharmaceutically acceptable carrier and optionally other therapeutic ingredients or adjuvants. Such additional therapeutic ingredients include, for example, i) Leukotriene receptor antagonists, ii) Leukotriene biosynthesis inhibitors, iii) corticosteroids, iv) H1 receptor antagonists, v) beta 2 adrenoceptor agonists, vi) COX-2 selective inhibitors, vii) statins, viii) non-steroidal anti-inflammatory drugs (“NSAID”), and ix) M2/M3 antagonists. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

Creams, ointments, jellies, solutions, or suspensions containing the compound of Formula I can be employed for topical use. Mouth washes and gargles are included within the scope of topical use for the purposes of this invention.

Dosage levels from about 0.001 mg/kg to about 140 mg/kg of body weight per day (or alternatively about 0.05 mg to about 7 g per patient per day) are useful in the treatment of conditions such as i) Pulmonary disorders such as asthma, chronic bronchitis, chronic obstructive pulmonary disease (COPD), adult respiratory distress syndrome, infant respiratory distress syndrome, cough, chronic obstructive pulmonary disease in animals, adult respiratory distress syndrome, and infant respiratory distress syndrome, ii) Gastrointestinal disorders such as ulcerative colitis, Crohn's disease, and hypersecretion of gastric acid, iii) Infectious diseases such as bacterial, fungal or viral induced sepsis or septic shock, endotoxic shock (and associated conditions such as laminitis and colic in horses), and septic shock, iv) Neurological disorders such as spinal cord trauma, head injury, neurogenic inflammation, pain, and reperfusion injury of the brain, v) Inflammatory disorders such as psoriatic arthritis, rheumatoid arthritis, ankylosing spondylitis, osteoarthritis, inflammation and cytokine-mediated chronic tissue degeneration, vi) Allergic disorders such as allergic rhinitis, allergic conjunctivitis, and eosinophilic granuloma, vii) Psychiatric disorders such as depression, memory impairment, and monopolar depression, viii) Neurodegenerative disorders such as Parkinson disease, Alzheimer's disease, acute and chronic multiple sclerosis, ix) Dermatological disorders such as psoriasis and other benign or malignant proliferative skin diseases, atopic dermatitis, and urticaria, x) Oncological diseases such as cancer, tumor growth and cancerous invasion of normal tissues, xi) Metabolic disorders such as diabetes insipidus, xii) Bone disorders such as osteoporosis, xiii) Cardiovascular disorders such as arterial restenosis, atherosclerosis, reperfusion injury of the myocardium, and xiv) Other disorders such as chronic glomerulonephritis, vernal conjunctivitis, transplant rejection and graft versus host disease, and cachexia—which are responsive to PDE4 inhibition. For example, inflammation may be effectively treated by the administration of from about 0.005 mg to 10 or 25 or 50 mg of the compound per kilogram of body weight per day, or alternatively about 0.25 mg to about 2.5 g per patient per day. Further, it is understood that the PDE4 inhibiting compounds of this invention can be administered at prophylactically effective dosage levels to prevent the above-recited conditions.

The amount of active ingredient that may be combined with the carrier materials to produce a single dosage form will vary depending upon the host treated and the particular mode of administration. For example, a formulation intended for the oral administration to humans may conveniently contain from about 0.25 mg to about 5 g of active agent, compounded with an appropriate and convenient amount of carrier material which may vary from about 5 to about 95 percent of the total composition. Unit dosage forms will generally contain between from about 0.01 mg to about 1000 mg of the active ingredient, typically 0.1 mg, 0.05 mg, 0.25 mg, 1 mg, 5 mg, 25 mg, 50 mg, 100 mg, 200 mg, 300 mg, 400 mg, 500 mg, 600 mg, 800 mg or 1000 mg.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

In practice, the compounds represented by Formula I, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula I, or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formula I. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.1 mg to about 500 mg of the active ingredient and each cachet or capsule preferably containing from about 0.1 mg to about 500 mg of the active ingredient.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol,), cyclodestrins, vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

The compounds and pharmaceutical compositions of this invention have been found to exhibit biological activity as PDE4 inhibitors. Accordingly, another aspect of the invention is the treatment in mammals of, for example, i) Pulmonary disorders such as asthma, chronic bronchitis, chronic obstructive pulmonary disease (COPD), adult respiratory distress syndrome, infant respiratory distress syndrome, cough, chronic obstructive pulmonary disease in animals, adult respiratory distress syndrome, and infant respiratory distress syndrome, ii) Gastrointestinal disorders such as ulcerative colitis, Crohn's disease, and hypersecretion of gastric acid, iii) Infectious diseases such as bacterial, fungal or viral induced sepsis or septic shock, endotoxic shock (and associated conditions such as laminitis and colic in horses), and septic shock, iv) Neurological disorders such as spinal cord trauma, head injury, neurogenic inflammation, pain, and reperfusion injury of the brain, v) Inflammatory disorders such as psoriatic arthritis, rheumatoid arthritis, ankylosing spondylitis, osteoarthritis, inflammation and cytokine-mediated chronic tissue degeneration, vi) Allergic disorders such as allergic rhinitis, allergic conjunctivitis, and eosinophilic granuloma, vii) Psychiatric disorders such as depression, memory impairment, and monopolar depression, viii) Neurodegenerative disorders such as Parkinson disease, Alzheimer's disease, acute and chronic multiple sclerosis, ix) Dermatological disorders such as psoriasis and other benign or malignant proliferative skin diseases, atopic dermatitis, and urticaria, x) Oncological diseases such as cancer, tumor growth and cancerous invasion of normal tissues, xi) Metabolic disorders such as diabetes insipidus, xii) Bone disorders such as osteoporosis, xiii) Cardiovascular disorders such as arterial restenosis, atherosclerosis, reperfusion injury of the myocardium, and xiv) Other disorders such as chronic glomerulonephritis, vernal conjunctivitis, transplant rejection and graft versus host disease, and cachexia—maladies that are amenable to amelioration through inhibition of the PDE4 isoenzyme and the resulting elevated cAMP levels—by the administration of an effective amount of the compounds of this invention. The term “mammals” includes humans, as well as other animals such as, for example, dogs, cats, horses, pigs, and cattle. Accordingly, it is understood that the treatment of mammals other than humans is the treatment of clinical correlating afflictions to those above recited examples that are human afflictions.

Further, as described above, the compound of this invention can be utilized in combination with other therapeutic compounds. In particular, the combinations of the PDE4 inhibiting compound of this invention can be advantageously used in combination with i) Leukotriene receptor antagonists, ii) Leukotriene biosynthesis inhibitors, iii) COX-2 selective inhibitors, iv) statins, v) NSAIDs, vi) M2/M3 antagonists, vii) corticosteroids, viii) H1 (histamine) receptor antagonists and ix) beta 2 adrenoceptor agonist.

Thus, for example, pulmonary disorders such as asthma, chronic bronchitis, chronic obstructive pulmonary disease (COPD), adult respiratory distress syndrome, infant respiratory distress syndrome, cough, chronic obstructive pulmonary disease in animals, adult respiratory distress syndrome, and infant respiratory distress syndrome can be conveniently treated with capsules, cachets or tablets each containing 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

Gastrointestinal disorders such as ulcerative colitis, Crohn's disease, and hypersecretion of gastric acid can be conveniently treated with capsules, cachets or tablets each containing 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

Infectious diseases such as bacterial, fungal or viral induced sepsis or septic shock, endotoxic shock (and associated conditions such as laminitis and colic in horses), and septic shock can be conveniently treated with capsules, cachets or tablets each containing 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

Neurological disorders such as spinal cord trauma, head injury, neurogenic inflammation, pain, and reperfusion injury of the brain can be conveniently treated with capsules, cachets or tablets each containing 0.25 mg, 0.5 mg, 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

Inflammatory disorders such as psoriatic arthritis, rheumatoid arthritis, ankylosing spondylitis, osteoarthritis, inflammation and cytokine-mediated chronic tissue degeneration can be conveniently treated with capsules, cachets or tablets each containing 0.25 mg, 0.5 mg, 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

Allergic disorders such as allergic rhinitis, allergic conjunctivitis, and eosinophilic granuloma can be conveniently treated with capsules, cachets or tablets each containing 0.25 mg, 0.5 mg, 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

Psychiatric disorders such as depression, memory impairment, and monopolar depression can be conveniently treated with capsules, cachets or tablets each containing 0.25 mg, 0.5 mg, 1 mg, 5 mg, 25 mg, 50 mg, 100 mg, 200 mg, 300 mg, 400mg, or 500mg of the active ingredient of the compound of the present application, or a pharmaceutically acceptable salt thereof, administered once, twice, or three times daily.

Neurodegenerative disorders such as Parkinson disease, Alzheimer's disease, acute and chronic multiple sclerosis can be conveniently treated with capsules, cachets or tablets each containing 0.25 mg, 0.5 mg, 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

Dermatological disorders such as psoriasis and other benign or malignant proliferative skin diseases, atopic dermatitis, and urticaria can be conveniently treated with capsules, cachets or tablets each containing 0.25 mg, 0.5 mg, 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

Oncological diseases such as cancer, tumor growth and cancerous invasion of normal tissues can be conveniently treated with capsules, cachets or tablets each containing 0.25 mg, 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

Metabolic disorders such as diabetes insipidus can be conveniently treated with capsules, cachets or tablets each containing 0.25 mg, 0.5 mg, 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

Bone disorders such as osteoporosis, cardiovascular disorders such as arterial restenosis, atherosclerosis, reperfusion injury of the myocardium, and other disorders such as chronic glomerulonephritis, vernal conjunctivitis, transplant rejection and graft versus host disease, and cachexia can be conveniently treated with capsules, cachets or tablets each containing 0.25 mg, 0.5 mg, 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

For enhancement of cognition (such as for of enhanced memory, learning, retention, recall, awareness and judgement), dosage levels from about 0.0001 mg/kg to about 50 mg/kg of body weight per day are useful or about 0.005 mg to about 2.5 g per patient per day. Alternatively, dosage levels from about 0.001 mg to 10 mg of the compound per kilogram of body weight per day, or alternatively about 0.05 mg to about 500 mg per patient per day.

The amount of active ingredient that may be combined with the carrier materials to produce a single dosage form will vary depending upon the host treated and the particular mode of administration. For example, a formulation intended for the oral administration to humans may conveniently contain from about 0.005 mg to about 2.5 g of active agent, compounded with an appropriate and convenient amount of carrier material. Unit dosage forms will generally contain between from about 0.005 mg to about 1000 mg of the active ingredient, typically 0.005, 0.01 mg, 0.05 mg, 0.25 mg, 1 mg, 5 mg, 25 mg, 50 mg, 100 mg, 200 mg, 300 mg, 400 mg, 500 mg, 600 mg, 800 mg or 1000 mg, administered once, twice or three times a day.

### ASSAYS DEMONSTRATING BIOLOGICAL ACTIVITY

### LPS and FMLP-Induced TNF-α and LTB₄ Assays in Human Whole Blood

Whole blood provides a protein and cell-rich milieu appropriate for the study of biochemical efficacy of anti-inflammatory compounds such as PDE4-selective inhibitors. Normal non-stimulated human blood does not contain detectable levels of TNF-α and LTB₄. Upon stimulation with LPS, activated monocytes express and secrete TNF-α up to 8 hours and plasma levels remain stable for 24 hours. Published studies have shown that inhibition of TNF-α by increasing intracellular cAMP via PDE4 inhibition and/or enhanced adenylyl cyclase activity occurs at the transcriptional level. LTB₄ synthesis is also sensitive to levels of intracellular cAMP and can be completely inhibited by PDE4-selective inhibitors. As there is little LTB₄ produced during a 24 hour LPS stimulation of whole blood, an additional LPS stimulation followed by fMLP challenge of human whole blood is necessary for LTB₄ synthesis by activated neutrophils. Thus, by using the same blood sample, it is possible to evaluate the potency of a compound on two surrogate markers of PDE4 activity in the whole blood by the following procedure.

Fresh blood was collected in heparinized tubes by venipuncture from healthy human volunteers (male and female). These subjects had no apparent inflammatory conditions and had not taken any NSAIDs for at least 4 days prior to blood collection. 500 μL aliquots of blood were pre-incubated with either 2 μL of vehicle (DMSO) or 2 μL of test compound at varying concentrations for 15 minutes at 37° C. This was followed by the addition of either 10 μL vehicle (PBS) as blanks or 10 μL LPS (1 μg/mL final concentration, #L-2630 (Sigma Chemical Co., St. Louis, Mo.) from E. coli, serotype 0111:B4; diluted in 0.1% w/v BSA (in PBS)). After 24 hours of incubation at 37° C., another 10 μL of PBS (blank) or 10 μL of LPS (1 μg/mL final concentration) was added to blood and incubated for 30 minutes at 37° C. The blood was then challenged with either 10 μL of PBS (blank) or 10 μL of fMLP (1 μM final concentration, #F-3506 (Sigma); diluted in 1% w/v BSA (in PBS)) for 15 minutes at 37° C. The blood samples were centrifuged at 1500×g for 10 minutes at 4° C. to obtain plasma. A 50 μL aliquot of plasma was mixed with 200 μL methanol for protein precipitation and centrifuged as above. The supernatant was assayed for LTB₄ using an enzyme immunoassay kit (#520111 from Cayman Chemical Co., Ann Arbor, Mich.) according to the manufacturer's procedure. TNF-α was assayed in diluted plasma (in PBS) using an ELISA kit (Cistron Biotechnology, Pine Brook, N.J.) according to manufacturer's procedure.

### Anti-Allergic Activity in Vivo

Compounds of the invention have been tested for effects on an IgE-mediated allergic pulmonary inflammation induced by inhalation of antigen by sensitized guinea pigs. Guinea pigs were initially sensitized to ovalbumin under mild cyclophosphamide-induced immunosuppression, by intraperitoneal injection of antigen in combinations with aluminum hydroxide and pertussis vaccine. Booster doses of antigen were given two and four weeks later. At six weeks, animals were challenged with aerosolized ovalbumin while under cover of an intraperitoneally administered anti-histamine agent (mepyramine). After a further 48 h, bronchial alveolar lavages (BAL) were performed and the numbers of eosinophils and other leukocytes in the BAL fluids were counted. The lungs were also removed for histological examination for inflammatory damage. Administration of compounds of the Examples (0.001-10 mg/kg i.p. or p.o.), up to three times during the 48 h following antigen challenge, lead to a significant reduction in the eosinophilia and the accumulation of other inflammatory leukocytes.

### SPA Based PDE Activity Assay Protocol

Compounds which inhibit the hydrolysis of cAMP to AMP by the type-IV cAMP-specific phosphodiesterases were screened in a 96-well plate format as follows:

In a 96 well-plate at 30° C. was added the test compound (dissolved in 2 μL DMSO), 188 mL of substrate buffer containing [2,8-³H] adenosine 3′, 5′-cyclic phosphate (cAMP, 100 nM to 50 μM), 10 mM MgCl₂, 1 mM EDTA, 50 mM Tris, pH 7.5. The reaction was initiated by the addition of 10 mL of human recombinant PDE4 (the amount was controlled so that ˜10% product was formed in 10 min.). The reaction was stopped after 10 min. by the addition of 1 mg of PDE-SPA beads (Amersham Pharmacia Biotech, Inc., Piscataway, N.J.). The product AMP generated was quantified on a Wallac Microbeta® 96-well plate counter (EG&G Wallac Co., Gaithersburg, Md.). The signal in the absence of enzyme was defined as the background. 100% activity was defined as the signal detected in the presence of enzyme and DMSO with the background subtracted. Percentage of inhibition was calculated accordingly. IC₅₀ value was approximated with a non-linear regression fit using the standard 4-parameter/multiple binding sites equation from a ten point titration.

The IC₅₀ values of the Examples disclosed here under were determined with 100 nM cAMP using the purified GST fusion protein of the human recombinant phosphodiesterase IVb (met-248) produced from a baculovirus/Sf-9 expression system.

1. Experimental Section

3.1. Preparation of Styrene Compound 2

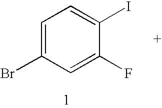

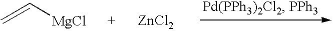

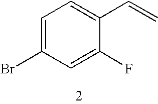

| Materials | MW | Amount | Moles |
|---|---|---|---|
| 1-Bromo-3-fluoro-4-iodobenzene | 300.89 | 5.0 kg | 16.62 |
| Vinyl magnesium chloride3 | 1.6 M in THF | 20.80 L | 33.24 |
| Zinc chloride | 0.5 M in THF | 33.2 L | 16.62 |
| Pd(PPh₃)₂Cl₂ | 701.89 | 200 g | 0.285 |
| PPh₃ | 262.29 | 149.5 g | 0.570 |
| Pentane | | 40 L | |

To a 72 L round bottomed flask was added zinc chloride THF solution (0.5 M, 33.2 L, 16.62 mol). The solution was cooled to −5° C. and vinyl magnesium chloride THF solution (1.6 M, 20.80 L, 33.24 mol) was added slowly, maintaining temperature at less than 20° C. Triphenylphosphine (149.5 g, 0.570 mol) was added, followed by Pd(PPh₃)₂Cl₂ (200 g, 0.285 mol). The mixture was stirred for 10 min, and 1-Bromo-3-fluoro-4-iodobezene was added. The reaction mixture was stirred at ambient temperature for 4-6 h until the reaction was complete by HPLC.
Mixing zinc chloride and vinyl magnesium chloride THF solutions was exothermic. The temperature was controlled by adjusting the addition rate and the cooling bath temperature.
The coupling reaction after the addition of aryl iodide (1) was slightly exorthermic. The temperature rose from 11° C. to 37° C. without a cooling bath in about 1 h and it cooled down thereafter.

The reaction mixture was quenched into a pre-cooled (0° C.) mixture of pentane (20 L), water (12 L), and concentrated HCl (1.0 L) in a 200 L extractor. The two layers were separated. The organic layer was diluted with pentane (20 L), washed with water (16 L), and concentrated under reduced pressure.
Compound 2 was quite volatile, and ˜20% was lost during rotavap concentration. Assay of the product before concentration normally gave product yield of 80-85%.

The product was further purified in this way: The residure was taken up with pentane (10 L). The resulting suspension was filtered. The solid was washed with pentane (1.0 L). The combined filtrate and wash were concentrated. The crude oil was purified by vacuum distillation at 0.1-0.2 mm Hg.
Purified product was light yellow with a boiling point of 45-50° C. at 0.1-0.2 mm Hg. Distillation recovery was ˜95%. Product was 93-95 wt %. The residue in the distillation pot was liquid at the end of distillation, but solidified upon cooling.
   1.2. Preparation of Cyclopropyl Aryl Bromide 3

1.2.1. Cyclopropanation

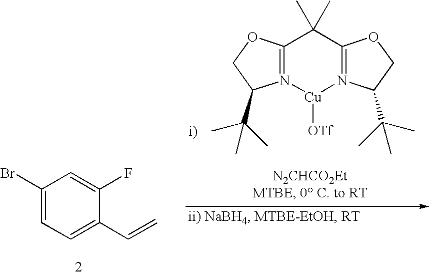

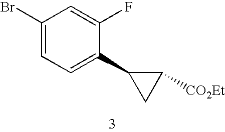

| Materials | MW | Amount | Moles |
|---|---|---|---|
| 4-Bromo-2-fluoro-1-vinylbenzene | 201.04 | 2.14 kg | 9.95 |
| (93.4%) | | (crude wt) | |
| Ethyl diazoacetate (88%) | 114.10 | 2.46 kg | 20.0 |
| | | (crude wt) | |
| Bisoxazoline ligand (96.5%) | 294.44 | 49.7 g | 0.163 |
| | | (crude wt) | |
| Copper(I) trifluoromethanesulfon- | 503.33 | 39.0 g | 0.0775 |
| ate benzene complex (2:1) | | | |
| MTBE | | 21.63 L | |
| Sodium borohydride (NaBH₄) | 37.83 | 105.2 g | 2.78 |
| Ethanol | | 5.12 L | |
| Aq. HCl (2 M) | | 6.11 L | 12.22 |
| Saturated aq. NaHCO₃ | | 3.33 L | |

A 5 L round bottom flask was charged with copper(I) trifluoromethanesulfonate benzene complex (39.0 g, 0.0775 mol) under a nitrogen atmosphere. The flask was charged with degassed MTBE (0.775 L) and cooled to 15° C. A solution of bisoxazoline ligand (49.7 g, 0.163 mol) in degassed MTBE (2.33 L) was added via cannula. The resulting suspension was stirred at 15-25° C. for 1 h and then allowed to stand for 30 min. The supernatant was filtered through an in-line filter to afford a deep green solution of catalyst.
Copper(I) trifluoromethanesulfonate benzene complex and the resulting copper complex are sensitive to oxygen and therefore should be handled under a nitrogen atmosphere.
The Cu(I) catalyst may be prepared in situ. In that case, 4-bromo-2-fluoro-1-vinylbenzene is added to a suspension of copper (I) trifluoromethanesulfonate and the bisoxazoline ligand in MTBE to afford a clear deep green solution. The reaction proceeds much more rapidly; however, a slightly lower selectivity (de and ee) is obtained.

A 72 L round bottom flask, equipped with a mechanical stirrer, a thermocouple, a nitrogen inlet, and an addition funnel, was charged with 4-bromo-2-fluoro-1 -vinylbenzene (2.00 kg assay wt, 9.95 mol). The flask was evacuated and filled with nitrogen three times. After cooling it to 0-5 ° C. (dry ice-acetone bath), a solution of the copper (I) complex, prepared above, was added. A solution of ethyl diazoacetate (38.7 g, 88%) in degassed MTBE (0.30 L) was added over 5 min, and the resulting mixture was aged for 10 min and assayed by GC.
Accumulation of ethyl diazoacetate should be avoided. Until formation of products is confirmed, the remainder of ethyl diazoacetate must not be added. The reaction mixture may need to be heated (20-30° C.) to initiate the conversion.

The remainder of ethyl diazoacetate (1.90 kg, 88%) in degassed MTBE (14.63 L) was slowly added over 7 h while maintaining the internal temperature at −2-13° C. After the addition was complete, the mixture was stirred at 0-5° C. for 2 h.
The addition of ethyl diazoacetate is very exothermic and generates a large volume of nitrogen gas. The progress of reaction must be checked to avoid the accumulation of ethyl diazoacetate. If either of gas evolution or heat generation ceases during the addition of ethyl diazoacetate, the reaction mixture might need to be heated (20-30° C.) to re-initiate the reaction. After the vinylbenzene is completely consumed, ethyl diazoacetate will react with itself to give diethyl maleate and diethyl flimarate, generating nitrogen gas and heat.
A slight excess (1.5 mol eq) of ethyl diazoacetate should be enough for complete conversion of the vinylbenzene. In the Prep Lab synthesis, however, a significant portion of the vinylbenzene remained. Thus, extra ethyl diazoacetate was added to obtain complete conversion.

A solution of ethyl diazoacetate (519 g) in degassed MTBE (3.6 L) was added over 90 min while maintaining the internal temperature at 0-14° C. The resulting mixture was stirred at 0-5° C. for 1 h and allowed to warm to 15° C.

A solution of NaBH₄ (105.2 g, 2.78 mol, approx. 0.6 mol eq with regard to dimers) in absolute ethanol (5.12 L) was added to the reaction solution, and the resulting mixture was stirred at 13-20 ° C. (20-25° C.) for 3.5 h.
The NaBH₄ reduction was slightly exothermic, and an ice-water bath may be used to cool the batch. The amount of NaBH₄ was based on the amount of dimers generated in the cyclopropanation. Reduction of the dimers gave diethyl succinate, which was confirmed by GC.

The reaction was cooled to 6° C. and quenched by addition of 2 M aq. HCl (6.11 L), while maintaining the batch temperature below 6° C. The resulting mixture was filtered and allowed to warm to 17° C. The organic layer was separated and washed with saturated aqueous NaHCO₃ (3.33 L). The chemical yield was 2418.9 g (85%).

1.2.2. Hydrolysis

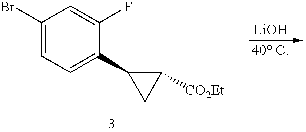

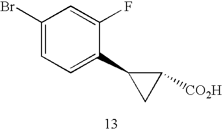

| Materials | MW | Amount | Moles |
|---|---|---|---|
| trans-Ethyl ester | 287.12 | 2.42 kg | 8.42 |
| Lithium hydroxide monohydrate | 41.96 | 817 g | 19.47 |
| MeOH | | 19.1 L | |
| Heptane | | 15.3 L | |
| MTBE | | 13.1 L | |
| Hexanes | | 9.88 L | |
| Aqueous HCl (2 M) | 9.28 L | 18.56 | |

A 72 L round bottom flask, equipped with mechanical stirrer, thermocouple, nitrogen inlet, and addition funnel, was charged with trans-ethyl ester (2.42 kg assay, crude solution from cyclopropanation). The solution was diluted with MeOH (13.8 L), and the flask was purged with nitrogen for 10 min. A solution of LiOH-H₂O (590 g, 13.8 mol) in H₂O (6.90 L) was slowly added. The temperature of the reaction mixture increased from 13° C. to 23° C. during the addition. An extra amount (227 g) of LiOH-H₂O was added, and the resulting mixture was heated to 38-40° C. for 4.5 h.
The starting ethyl ester was first converted to the corresponding methyl ester by solvolysis with methanol and then to the carboxylic acid.
trans-Esters are more reactive toward basic methanol or NaOH than cis-esters. The diastereomeric excess of the product (carboxylic acid) should be much higher than that of the starting material. The stirring was continued until the level of cis-acid started to increase more rapidly than trans-acid did. The final diastereomeric excess of the product was typically 97% (de).

The reaction was cooled to 20° C., transferred to an extractor cylinder, and diluted with H₂O (28.7 L) and heptane (5.42 L) with stirring. The aqueous layer was separated, filtered through an in-line filter, and washed with heptane (9.88 L). Hexanes (9.88 L) and MTBE (13.1 L) were added, and the resulting mixture was cooled to 0-10° C. Aqueous HCl (10.7 L, 2 M) was added while maintaining the temperature below 10° C. with stirring, and the mixture was allowed to warm to 17° C. with stirring. The yield was 2052.6 g (94%).

Solvent was evaporated, and the resulting solid was dried under reduced pressure. The dried solid was dissolved in MeOH (5.31 L). H₂O (2.92 L) was slowly added while maintaining the batch temperature below 23° C. A slurry of carboxylic acid (40 g) in MeOH/H₂O (100 mL/55 mL) was added as seeding crystals. The resulting mixture was stirred at 23° C. for 10 min. H₂O (15.5 L) was added over 80 min while maintaining the batch temperature below 24° C., and the slurry was stirred at 22-24° C. for 2 h. The solid was collected by filtration, washed with H₂O (10.7 L), and dried under a flow of nitrogen to afford carboxylic acid as pale yellow solids (2019 g assay wt).

1.2.3. Esterification

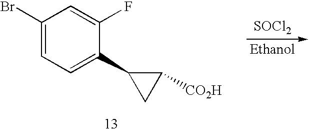

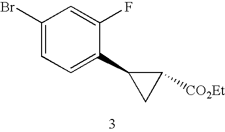

| Materials | MW | Amount | Moles |
|---|---|---|---|
| Aryl bromide acid 13 | 259.08 | 2.19 kg (97.3%) | 8.22 |
| Thionyl chloride | 118.97 | 0.64 L | 8.77 |
| EtOH | 57.1 | 9.0 L | |
| Na2CO3.H2O | 124.00 | 1.92 kg | 15.5 |
| Toluene | | 14.0 L | |

To a stirred solution of the arylboronic acid 13 (2.19 kg) in ethanol (9.0 L) at 4° C. in a 22 L round bottom flask fitted with stirrer and temperature probe, was added thionyl chloride (0.64 L) through a dropping funnel over 1 h. After the addition was complete, the solution was stirred for 1 h at 11° C. and then at 40-45° C. for 2 h. The solution was cooled to 20° C., and toluene (9.0 L) was added. A 100 L jacketed cylinder, fitted with stirrer and temperature probe, was charged with water (12 L) and sodium carbonate monohydrate (1.92 kg). The sodium carbonate solution was cooled to 10° C. and the reaction batch was transferred through a vacuum line into the 100 L cylinder with stirring over 20 min at 15-20° C. The two phases was separated, and the aqueous phase was back extracted with toluene (5.0 L). The organic phases were combined and concentrated. The resulting solution was used directly in the next step reaction, and the assay yield was 95%.

3.3. Preparation of Amide Boronic Acid

3.3.1. Preparation of Boronic Acid Pinacol Ester

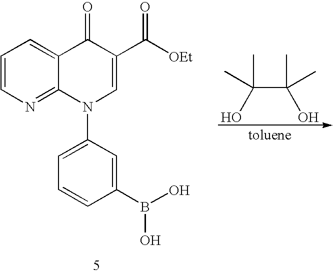

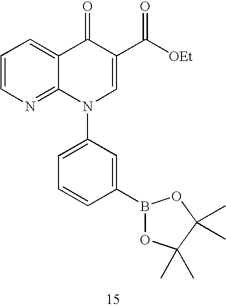

| Materials | MW | Amount | Moles |
|---|---|---|---|
| Boronic acid 5 | 338.12 | 3.01 kg (71 wt %) | 6.33 |
| Pinacol | 118.17 | 0.83 kg | 6.90 |
| Toluene | | 30.5 L | |
| Hexane | | 32.0 L | |

To a stirred suspension of the boronic acid 5 (3.01 kg) in toluene (30.0 L) at ambient temperature in a 50 L flask, was added pinacol (0.83 kg) through a powder funnel. Toluene (0.5 L) was used to rinse in any remaining material on the funnel. The mixture was heated at reflux temperature for 3 h during which time water was removed by azeotropic distillation (collected with a Dean-Stark trap).
Initial reflux temp was 83.5° C., which rose to 106° C. over 3 h. The resulting solution was allowed to cool overnight during which time product crystallized.
Acid 22 less than 0.2 LCAP. ¹H NMR disappearance of B-OH in spectrum

The reaction mixture was concentrated at reduced pressure to ˜12 L, and hexane (24 L) was added. The suspension was stirred for 2 h at ambient temperature. The product was isolated by filtration, and the filter cake was washed with hexane (2×4 L). The product was dried on the filter overnight, transferred to a vacuum oven on trays, and dried at 35° C. under a stream of nitrogen to give product (2.55 kg, 98.0 wt %) in 95.2% yield. Product loss in the filtrate was 3.2%.

1.3.2. Amidation

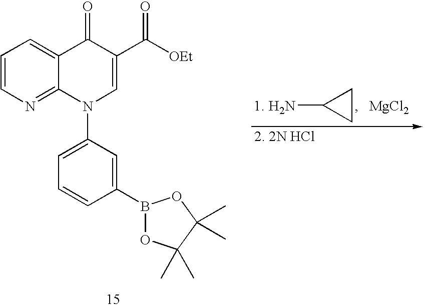

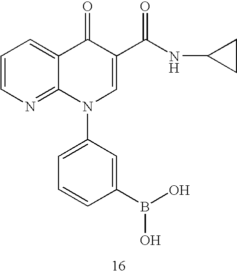

| Materials | MW | Amount | Moles |
|---|---|---|---|
| Ester 15 | 420.19 | 2.90 kg (98 wt %) | 6.90 |
| MgCl2 | 95.21 | 0.57 kg | 5.90 |
| Cyclopropylamine | 57.09 | 32.40 L | 33.94 |
| DMAc | | 10.8 L | |
| 2.5 M HCl | | 55.0 L | |

To a stirred suspension of the pinacol ester 15 (2.90 kg) in DMAc (10 L) in a 22 L round bottom flask, fitted with stirrer and temperature probe, was added MgCl₂ (0.57 kg) in one portion. The temperature of the batch rose from 24° C. to 38° C. The suspension was degassed (3×nitrogen/vacuum purge), and cyclopropylamine (2.4 L) was added over 5 min. The temperature of the batch rose to 44.5° C., and a solution was obtained. The solution was stirred at 40-45° C. for 3 h.

To a 100 L jacketed cylinder, fitted with stirrer and temperature probe, was charged 2.5 N HCl (55 L). The batch was transferred under vacuum to the 100 L cylinder over 1 hat 15-18° C. The transfer line was rinsed with DMAc (0.8 L), and water (4 L) was added. The suspension was stirred at 15° C. for 2 h. The product was isolated by filtration and dried at reduced pressure.
Filtration was very slow and the batch was split into two filter pots. The batch was washed with water.
The drying process was extremely long, but product contains water may be used in the Suzuki coupling. The isolated yield for this step was ˜93%.

3.4. Suzuki Coupling

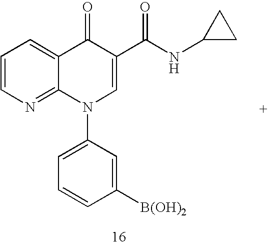

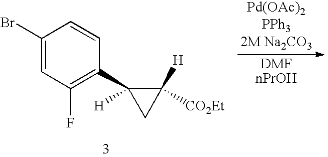

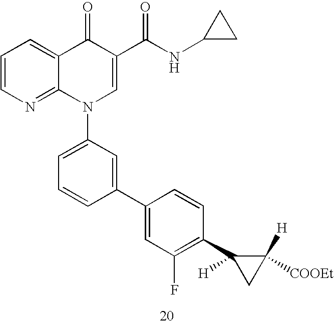

| Materials | MW | Amount | Moles |
|---|---|---|---|
| Aryl bromide 3 | 287.13 | 1.40 kg (67 wt %) | 3.27 |
| Boronic acid 16 | 349.15 | 1.68 kg (72.5 | 3.48 |
| | | wt %) | |
| Pd(OAc)₂ | 224.49 | 14.9 g | 0.066 |
| PPh₃ | 262.28 | 52.2 g | 0.20 |
| DMF | | 17.2 L | |
| 1-propanol | | 17.2 L | |
| Na₂CO₃.H₂O | 124.00 | 1.44 kg | 11.6 |

A 100 L, four-necked flask, equipped with mechanical stirrer, condenser with N₂ inlet, thermocouple, and stopper, was purged with N₂ and charged with DMF (8 L) and nPrOH (8 L), followed by Pd(OAc)₂ (14.9 g) and PPh₃ (52.2 g). The solids were washed in with DMF (4 L) and nPrOH (4 L).
The solids are carefully washed from the flask walls because any Pd(OAc)₂ adhering to the walls will become black during the course of the reaction.

The mixture was stirred for 15 min at 18-23° C. To the flask was added boronic acid (1.68 kg) and aryl bromide (1.40 kg), followed by DMF (2.7 L), nPrOH (2.7 L), and a 2 M solution of Na₂CO₃.H₂O (1.44 kg) in H₂O (sufficient to make 5.79 L of solution). The reaction mixture was heated to 70° C. using a steam pot.

After 4 h, HPLC showed 0.3 A% aryl bromide. Heating was stopped, and the mixture was slowly cooled to 22° C. over 2 h with gentle stirring. Water (14.7 L) was added over 30 min, and the mixture was cooled to 0-5° C. (1 h). The slurry was filtered, and the cake was washed with cold 1: 1:2 DMF/nPrOH/H₂O (10 L), followed by H₂O (30 L). The cake was dried with a N₂ sweep under reduced pressure to give 1.61 kg of light yellow solid.
The product was 93.0 wt %, 96.2 A% (89.7% yield). The palladium level was 980 ppm. HPLC of the filtrate and first wash showed 28 g, 1.7%.

1.5. Hydrolysis and Pd Removal

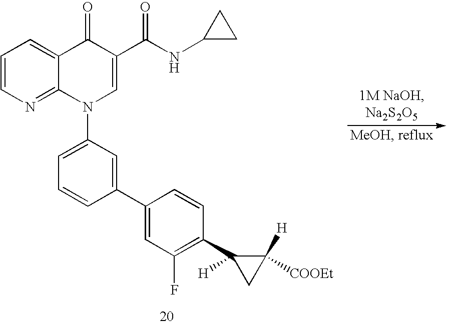

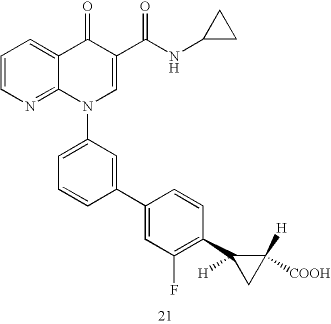

| Materials | MW | Amount | Moles |
|---|---|---|---|
| Suzuki product | 511.54 | 2.63 kg | 5.14 |
| Aq. NaOH (1 M) | 40.00 | 15.4 L | 15.40 |
| Na₂S₂O₅ | 190.10 | 97.7 g | 0.51 |
| MeOH | | 26.2 L | |
| Aq. HCl (1 M) | 36.46 | 16.9 L | 16.9 |
| THF | | 20.6 L | |

A 72 L round bottom flask, equipped with mechanical stirrer, thermocouple, nitrogen inlet, and reflux condensor, was charged with Suzuki product (2.63 kg assay, Pd=299 ppm), Na₂S₂O₅ (97.7 g), and MeOH (26.2 L). Aq. NaOH (15.4 L) was added, and the mixture was heated to reflux for 2 h.

After the Suzuki product was completely consumed, the reaction mixture was cooled to 20° C. and aged at that temperature for 3-12 h. The resulting hazy solution was filtered through a pad of Celite (2.0 kg) to remove residual palladium and impurities. The Celite cake was rinsed with MeOH/H₂O (2/1, 14.0 L).
The filtration removes a significant amount of a dimer byproduct (24) and palladium. Aging at 20° C. needs to be continued until the amount of the dimer product in the supernatant is reduced to a satisfactory level. A small portion of the reaction mixture was filtered by a syringe filter and assayed the level.
The filtration was very slow. Addition of carbon or other resin during the hydrolysis or during the room temperature age may aid the filtration and removal of Pd, which will be studied further.
The sodium salt of Compound of Formula (21)) is a crystalline compound and may precipitate during the filtration. Therefore, the Celite cake might need to be thoroughly rinsed with MeOH/H₂O to ensure the product is completely eluted into the filtrate. The filtrate and washes were combined.
Assay at this point indicated 2.43 kg free acid (98% yield).

The combined solutions were added slowly into a mixture of THF (20.6 L) and aq. 1 M HCl (16.9 L) over 2 h, maintainning the temperature at 20-25° C. The resulting slurry was aged at 22-24° C. for 1 h. The solid was collected by filtration, washed with H₂O (12.0 L), and partially dried to afford wet cake (4.6 kg).
Drying in the filter pot under N2/vacuum was very slow. Oven drying at elevated temperature should be studied in the future.
The wet cake was 51.4 wt %. Assay wt.: 2.36 kg (95.2% overall yield).
The Pd level was 56 ppm (based on dried weight). A repeat of the process reduced the level to 19 ppm. When repeating the process the third time, 5 wt % charcoal was added during the heating with NaOH in methanol. The product had a Pd level of 6 ppm. Further studies are needed to obtain a robust Pd removal process.

1.5. Formation of Sodium Salt

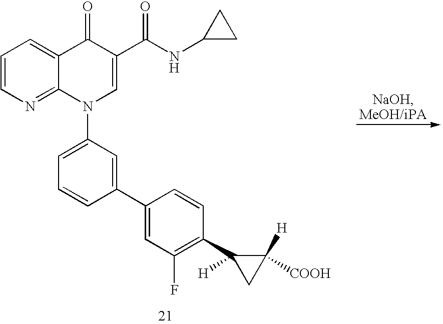

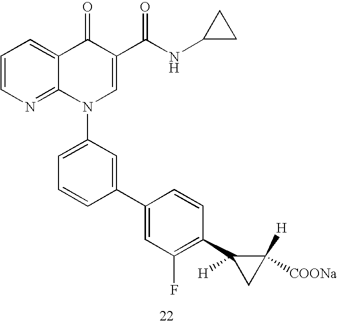

| Materials | MW | Amount | Moles |
|---|---|---|---|
| Formula 21 (free acid) | 483.49 | 2.63 kg (82.6 | 4.49 |
| | | wt %) | |
| Aq. NaOH (10.0 N) | 40.00 | 471 mL | 4.71 |
| MeOH | | 4.88 L | |
| 2-PrOH | | 52.1 L | |

A 100 L round bottom flask, equipped with mechanical stirrer, thermocouple, and nitrogen inlet, was charged with acid (2.63 kg, 82.6 wt %), MeOH (4.88 L), and H20 (4.24 L). Aqueous NaOH (471 mL, 10.0 N) was added, and the mixture was heated to 40° C. until most of solids dissolved. 2-PrOH (52.1 L) was added, and the mixture was allowed to cool to 26° C. and age at 22-26° C.
2-PrOH is preferably added slowly to prevent the sodium salt from coming out as oil. During the prep lab prep, a small amount of product oiled out. Consequently, the mixture was heated at ˜70° C. for ˜2 h to convert the oil to crystalline solid before cooling to 22° C. The concentration of product in the supernatant at the end of the age at 22° C. was typically ˜2 mg/mL. The crystallization was slow and normally took greater than 3 h to complete.

The solid was collected by filtration, washed with 1: 10 H₂O/IPA (5.5 L), 1:15 H₂O/IPA (5.0 L), and IPA (5.0 L×2), and dried under a flow of nitrogen to afford 2.02 kg of an off-white solid.
Product had 4 ppm Pd. Product loss in the filtrate and washes was 127 g and 29 g respectively.
   Experimental for Characterization of Salt
   X-Ray Powder Diffraction

X-ray diffraction patterns were measured using a Panalytical X'Pert Pro with a Cu LFF source (Cu K-alpha−wavelength=1.54187) at a generator power of 40 kV and 50 mA from 2-40 degrees 2-theta.

C-13 SSNMR

The solid-state carbon-13 NMR spectra were obtained on a Bruker DSX 500 WB NMR system using a Bruker 4 mm H/X/Y CPMAS probe. The carbon-13 NMR spectra utilized proton/carbon-13 cross-polarization magic-angle spinning with variable-amplitude cross polarization, total sideband suppression, and SPINAL decoupling at 100 kHz. The samples were spun at 10.0 kHz, and a total of 1024 scans were collected with a recycle delay of 5 seconds. A line broadening of 10 Hz was applied to the spectra before FT was performed. Chemical shifts are reported on the TMS scale using the carbonyl carbon of glycine (176.03 p.p.m.) as a secondary reference.

19-F SSNMR

The solid-state fluorine-19 NMR spectra were obtained on a Bruker DSX 500 WB NMR system using a Bruker 4 mm H/F/X CPMAS probe. The fluorine-19 NMR spectra utilized proton/fluorine-19 cross-polarization magic-angle spinning with variable-amplitude cross polarization, and TPPM decoupling at 62.5 kHz. The samples were spun at 15.0 kHz, and a total of 256 scans were collected with a recycle delay of 5 seconds. A line broadening of 10 Hz was applied to the spectrum before FT was performed. Chemical shifts are reported using poly(tetrafluoroethylene) (Teflon®) as an external secondary reference which was assigned a chemical shift of −122 ppm.

Raman Spectroscopy

The data was acquired using a Bruker RFS 100/S Raman spectrometer. Samples were analyzed using 250 mW laser strength with a total of 64 scans at 4 cm⁻¹ resolution. The samples were measured a minimum of four times at 2-mm diameter metal sample holders and averaged. Peak position was verified using sulfur (Anachemia AC-8734). The spectra were normalized within the region of interest for comparative purposes.

### DISCUSSION

Overview

Disclosed is a PDE4 inhibitor of the Formula (22) as well as process for making same. One of the reaction step is the stereoselective cyclopropanation of 2 to provide 3. Excellent diastereoselectivity (93:7) and enantioselectivity (>98% ee) were obtained for the desired stereoisomer. A non-cryogenic reaction was discovered for the preparation of the styrene derivative (2). An improved process for the synthesis of the boronic acid piece (5) from 4 is disclosed. Boronic acid 5 was converted to the corresponding amide 6, which was then coupled with the cyclopropyl compound 3. After hydrolysis, the coupled product was converted to the compound of Formula (21) (the free acid). A superior salt of the compound of Formula (21) (the sodium salt) was identified. The crystalline sodium salt was characterized by XRPD, DSC, and TGA. 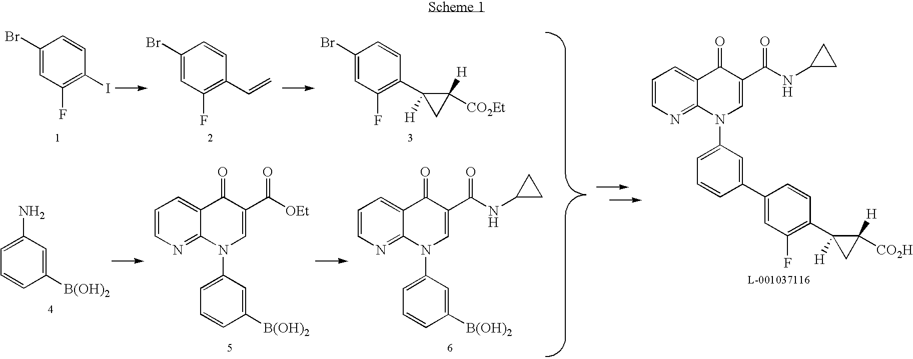
Remarks
2.1. Cyclopropanation and Purification of Compound 3

An improved Evans cyclopropanation protocol was used for this synthesis using the Cu catalyst prepared from copper (I) triflate and chiral ligand 10. Other ligands and Rh catalysts were tried but all afforded lower diastereoselectivity. The major by-products from the reaction were the cis-isomer, 11 and 12 from the dimerization of ethyl diazoacetate. Solvent plays a significant role in enantioselectivity, diastereoselectivity, and formation of the dimer impurities. As shown in Table 1, a variety of solvents, including coordinating and non-coordinating ones, gave good to excellent conversions (74-98%), except for THF (45%). The diastereoselectivity varied from 80:20 (trans:cis, 1,2-dichloroethane) to 93:7 (trans:cis, MTBE), and ee varied from 85% (1,2-dichloroethane) to 99% (many solvents including MTBE). MTBE gave the best results and was used as the solvent for our first GMP campaign. A significant amount of precipitate was formed when the catalyst was prepared in MTBE. In early studies, this precipitate was removed by filtration prior to the cyclopropanation. However, conversions and ethyl diazoacetate accumulation varied from batch to batch. The situation was greatly improved by generation of the catalyst in situ without filtration. The solid catalyst was completely dissolved after the addition of styrene, giving a clear solution before addition of ethyl diazoacetate. Similar diastereoselectivity and enantioselectivity were obtained. In the prep lab, the cyclopropanation reaction was run in two batches. The first batch used the procedure with the solid catalyst removed and 2.4 kg (assayed, 85% yield after NaBH4 treatment, see below) of 3 was obtained with a trans/cis ratio of 92:8 and 98.8% ee for the trans. The conversion for the reaction was only 95% with 2.0 equiv of ethyl diazoacetate used. The second batch used the procedure with in situ generated catalyst without solid removal. Complete conversion was observed with the use of 1.5 equiv of ethyl diazoacetate. Again, 2.4 kg (assayed, 85% yield after NaBH4 treatment) of 3 was obtained with a trans/cis ratio of 88:12 and 98.9% ee for the trans.

TABLE 1

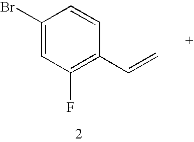

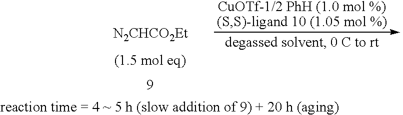

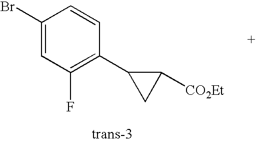

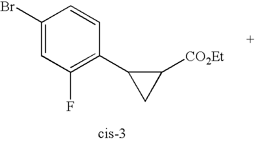

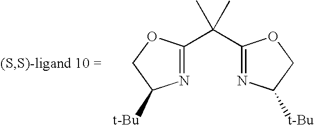

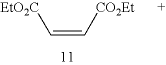

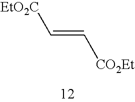

| solvent | trans-3 | cis-3 | trans + cis | 2 | 11 | 12 | ee (trans) | ee (cis) |
|---|---|---|---|---|---|---|---|---|
| CHCl₃ | 84% | 11% | 95% (99:12) | 0% | 33% | 9% | 99% | 96% |
| (alumina; K₂CO₃) | | | | | | | | |
| 1,2-dichloroethane | 59% | 15% | 74% (80:20) | 21% | 9% | 38% | 85% | 94% |
| (MS 4A) | | | | | | | | |
| toluene | 71% | 6% | 77% (92:8) | 18% | 55% | 8% | 99% | 97% |
| (MS 4A) | | | | | | | | |
| 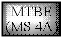 | 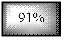 | 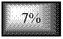 | 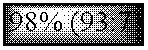 | 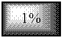 | 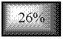 | 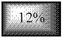 | 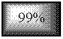 | 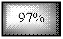 |
| ethylacetate | 86% | 11% | 97% (89:11) | 0% | 21% | 17% | 99% | 97% |
| (none) | | | | | | | | |
| THF | 40% | 5% | 45% (89:11) | 50% | 9% | 0% | 93% | 96% |
| (NS 4A) | | | | | | | | |
| iPAC | 87% | 10% | 97% (90:10) | 1% | 19% | 16% | ND | ND |
| (none) | | | | | | | | |
| a,a,a-trifluorotoluene | 79% | 8% | 87% (91:9) | 10% | 38% | 11% | ND | ND |
| (none) | | | | | | | | |

2.2. Amidation

The naphthyridone boronic acid 5 contained high levels (10-20% by weight) of residual water. Direct cyclopropylamidation of 5 by cyclopropylamine in either DMF or DMAc at 40-50° C. proved to be problematical, and considerable amounts of the acid 22 (Scheme 3) were formed. Direct drying of the boronic acid raised concerns of boronic anhydride formation. Also, the relative insolubility of boronic acids 5 and 16 made it difficult to obtain pure samples for assay purposes. Formation of pinacol ester 15 from 5 in refluxing toluene, with water removed using a Dean-Stark trap, followed by addition of hexane as an anti solvent gave 15 in greater than 95% isolated yield. Treatment of 15 with cyclopropylamine in either DMF or DMAc at 40-50° C. in the presence of MgCl₂ gave 16 in 90-95% isolated yield after quenching into dilute HCl. The acid impurity 22 was typically controlled at <2%. It was necessary to degas the slurry of 15 and MgCl₂ prior to addition of cyclopropylamine to minimize formation of phenol 21 to less than 0.5 A%.

The compound of Formula 16 was obtained in about 94% yield.

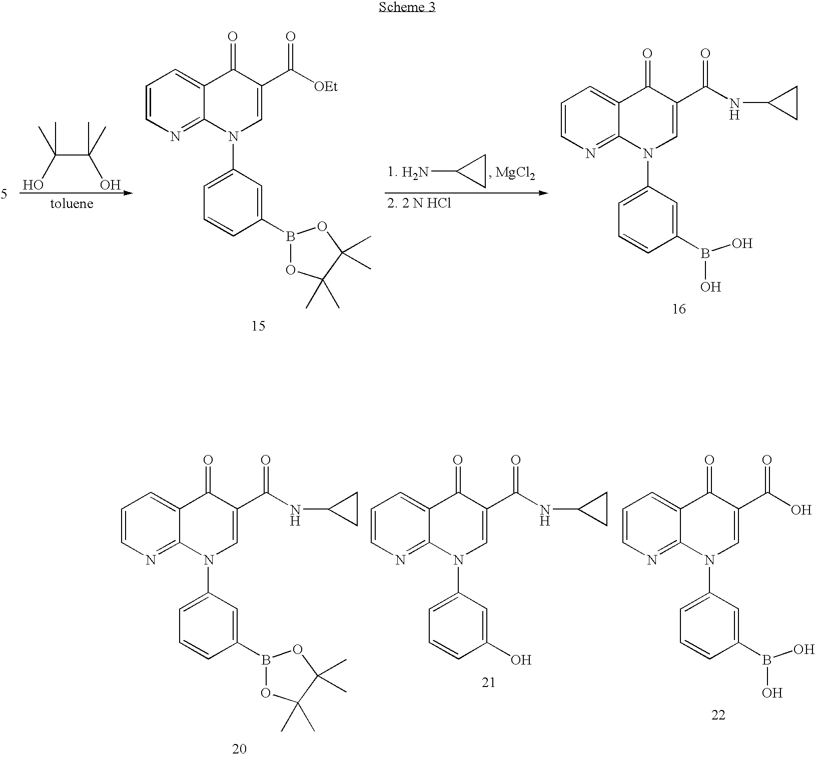

Other variations or modifications, which will be obvious to those skilled in the art, are within the scope and teachings of this invention. This invention is not to be limited except as set forth in the following claims.

What is claimed is:

1. A compound of the Formula (22)

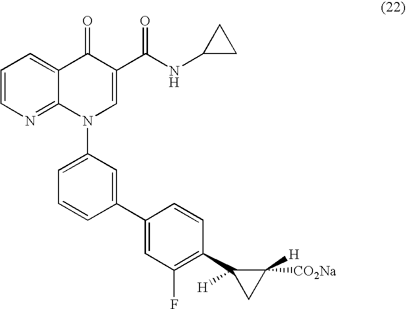

2. A pharmaceutical composition comprising a compound according to claim 1 and a pharmaceutically acceptable carrier.

3. The pharmaceutical composition according to claim 2, further comprising a Leukotriene receptor antagonist, a Leukotriene biosynthesis inhibitor, an M2/M3 antagonist, a corticosteroid, an H1 receptor antagonist or a beta 2 adrenoceptor agonist.

4. The pharmaceutical composition according to claim 2, further comprising a COX-2 selective inhibitor, a statin, or an NSAID.

5. A method of treatment or prevention of asthma, chronic bronchitis, chronic obstructive pulmonary disease (COPD), eosinophilic granuloma, psoriasis and other benign or malignant proliferative skin diseases, endotoxic shock (and associated conditions such as laminitis and colic in horses), septic shock, ulcerative colitis, Crohn's disease, reperfusion injury of the myocardium and brain, inflammatory arthritis, osteoporosis, chronic glomerulonephritis, atopic dermatitis, urticaria, adult respiratory distress syndrome, infant respiratory distress syndrome, chronic obstructive pulmonary disease in animals, diabetes insipidus, allergic rhinitis, allergic conjunctivitis, vernal conjunctivitis, arterial restenosis, atherosclerosis, neurogenic inflammation, pain, cough, rheumatoid arthritis, ankylosing spondylitis, transplant rejection and graft versus host disease, hypersecretion of gastric acid, bacterial, fungal or viral induced sepsis or septic shock, inflammation and cytokine-mediated chronic tissue degeneration, osteoarthritis, cancer, cachexia, muscle wasting, depression, memory impairment, monopolar depression, acute and chronic neurodegenerative disorders with inflammatory components, Parkinson disease, Alzheimer's disease, spinal cord trauma, head injury, multiple sclerosis, tumour growth and cancerous invasion of normal tissues comprising the step of administering a therapeutically effective amount, or a prophylactically effective amount, of the compound according to claim 1.

6. A method of enhancing cognition in a subject comprising administering a safe cognition enhancing amount of compound according to claim 1.

7. A crystalline compound of claim 1.

8. A crystalline form of the compound of structural formula (21)

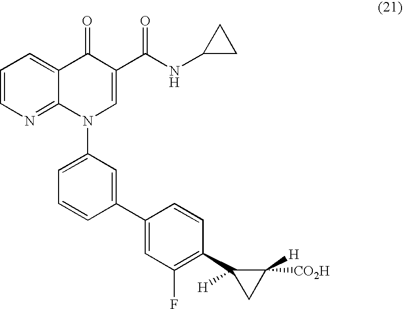

9. A pharmaceutical compositions comprising crystalline compound of structural formula (21)or formula (22) according to claim 7 or claim 8 a pharmaceutically acceptable carrier.

10. A pharmaceutical composition according to claim 9 further comprising a Leukotriene receptor antagonist, a Leukotriene biosynthesis inhibitor, an M2/M3 antagonist, a corticosteroid, an H1 receptor antagonist or a beta 2 adrenoceptor agonist.

11. A pharmaceutical composition according to claim 9 further comprising a COX-2 selective inhibitor, a statin, or an NSAID.

12. A method of treatment or prevention of asthma, chronic bronchitis, chronic obstructive pulmonary disease (COPD), eosinophilic granuloma, psoriasis and other benign or malignant proliferative skin diseases, endotoxic shock (and associated conditions such as laminitis and colic in horses), septic shock, ulcerative colitis, Crohn's disease, reperfusion injury of the myocardium and brain, inflammatory arthritis, osteoporosis, chronic glomerulonephritis, atopic dermatitis, urticaria, adult respiratory distress syndrome, infant respiratory distress syndrome, chronic obstructive pulmonary disease in animals, diabetes insipidus, allergic rhinitis, allergic conjunctivitis, vernal conjunctivitis, arterial restenosis, atherosclerosis, neurogenic inflammation, pain, cough, rheumatoid arthritis, ankylosing spondylitis, transplant rejection and graft versus host disease, hypersecretion of gastric acid, bacterial, fungal or viral induced sepsis or septic shock, inflammation and cytokine-mediated chronic tissue degeneration, osteoarthritis, cancer, cachexia, muscle wasting, depression, memory impairment, monopolar depression, acute and chronic neurodegenerative disorders with inflammatory components, Parkinson disease, Alzheimer's disease, spinal cord trauma, head injury, multiple sclerosis, tumour growth and cancerous invasion of normal tissues comprising the step of administering a therapeutically effective amount, or a prophylactically effective amount, of the crystalline compound of structural formula (21) or (22) according to claim 7 or claim 8.

13. A method of enhancing cognition in a subject comprising administering a safe cognition enhancing amount of crystalline compound of structural formula (21) or (22) according to claim 7 or claim 8.

14. The crystalline sodium salt of the compound structural formula (22) according to claim 1 characterized by diffraction peaks obtained from the X-ray powder diffraction pattern corresponding to d-spacings of 10.05, 5.16, 8.76 angstroms.

15. The crystalline sodium salt of the compound structural formula (22) according to claim 1 characterized by diffraction peaks obtained from the X-ray powder diffraction pattern corresponding to d-spacings of 3.83, 4.11, 5.95 angstroms.

16. The crystalline sodium salt of the compound structural formula (22) according to claim 1 characterized by diffraction peaks obtained from the X-ray powder diffraction pattern corresponding to d-spacings of 17.67, 5.57, 4.90 angstroms.

17. The crystalline sodium salt of the compound structural formula (22) according to claim 1 characterized by characteristic diffraction peaks obtained from the X-ray powder diffraction pattern corresponding to a d-spacing of 10.05 angstroms.

18. The crystalline sodium salt of the compound structural formula (22) according to claim 1 characterized by the X-ray powder diffraction pattern of FIG. 1.

19. The crystalline sodium salt of the compound structural formula (22) according to claim 1 characterized by a solid-state carbon-13 CPMAS nuclear magnetic resonance spectrum showing signals at 169.1, 120.8, and 46.5 ppm.

20. The crystalline sodium salt of the compound structural formula (22) according to claim 1 characterized by a solid-state carbon-13 CPMAS nuclear magnetic resonance spectrum showing signals at 159.0, 150.9, and 40.7 ppm.

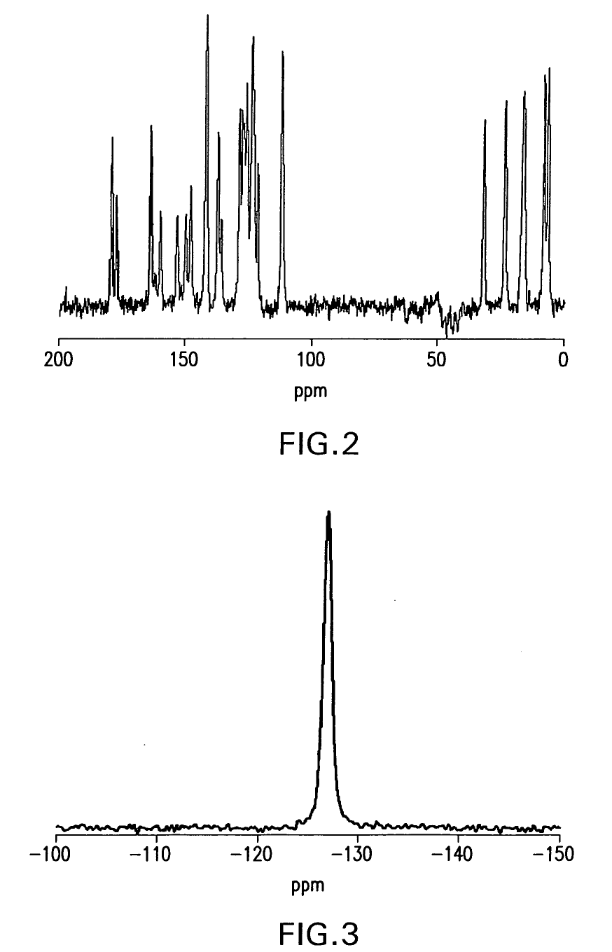

21. The crystalline sodium salt of the compound structural formula (22) according to claim 1 characterized by the solid-state carbon-13 CPMAS nuclear magnetic resonance spectrum of FIG. 2.

22. The crystalline sodium salt of the compound structural formula (22) according to claim 1 characterized by a solid-state fluorine-19 MAS nuclear magnetic resonance spectrum showing signal at −126.8 ppm,

23. The crystalline sodium salt of the compound structural formula (22) according to claim 1 characterized by the solid-state fluorine-19 MAS nuclear magnetic resonance spectrum of FIG. 3.

24. The crystalline sodium salt of the compound structural formula (22) according to claim 1 further characterized by absorption bands obtained from the Raman spectrum at 1625, 1609, 1600 wavenumbers (cm⁻¹).

25. The crystalline sodium salt of the compound structural formula (22) according to claim 1 further characterized by absorption bands obtained from the Raman spectrum at 723 wavenumbers (cm⁻¹).

26. The crystalline sodium salt of the compound structural formula (22) according to claim 1 is further characterized by absorption bands obtained from the Raman spectrum at 1294, 1281, 1000 wavenumbers (cm⁻¹).

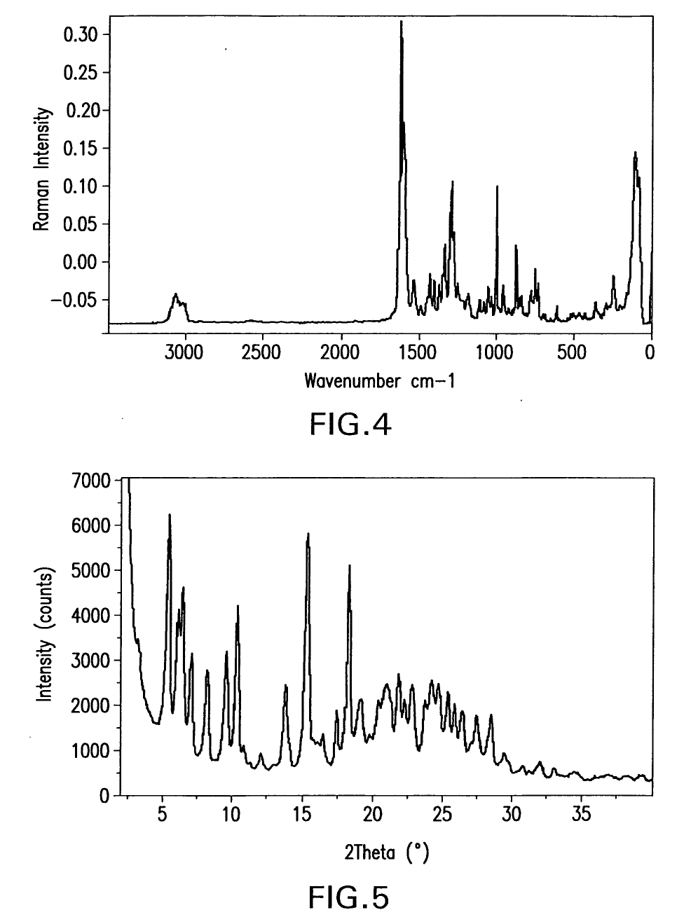

27. The crystalline sodium salt of the compound structural formula (22) according to claim 1 is further characterized by the Raman spectrum shown in FIG. 4.

28. The crystalline free acid of the compound structural formula (21) according to claim 8 characterized by diffraction peaks obtained from the X-ray powder diffraction pattern corresponding to d-spacings of 16.37, 5.79, 4.85 angstroms.

29. The crystalline free acid of the compound structural formula (21) according to claim 8 characterized by diffraction peaks obtained from the X-ray powder diffraction pattern corresponding to d-spacings of 13.81, 8.51, 14.49 angstroms.

30. The crystalline free acid of the compound structural formula (21) according to claim 8 characterized by characterized by diffraction peaks obtained from the X-ray powder diffraction pattern corresponding to d-spacings of 12.63, 10.78, 9.21 angstroms.

31. The crystalline free acid of the compound structural formula (21) according to claim 8 characterized by further characterized by the X-ray powder diffraction pattern of FIG. 5.

32. The crystalline free acid of the compound structural formula (21) according to claim 8 characterized by characterized by a solid-state carbon-13 CPMAS nuclear magnetic resonance spectrum showing signals at 23.2, 128.3, and 148.6 p.p.m.

33. The crystalline free acid of the compound structural formula (21) according to claim 8 characterized by characterized by a solid-state carbon-13 CPMAS nuclear magnetic resonance spectrum showing signals at 7.4, 181.6, and 120.9 p.p.m.

34. The crystalline free acid of the compound structural formula (21) according to claim 8 characterized by characterized by a solid-state carbon-13 CPMAS nuclear magnetic resonance spectrum showing signals at 179.2, 163.7, and 110.4 p.p.m.

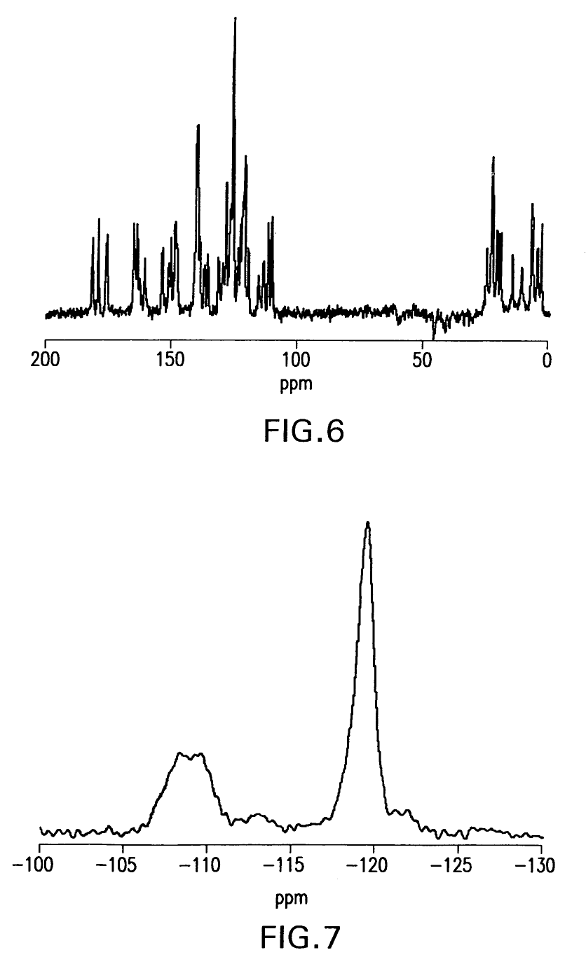

35. The crystalline free acid of the compound structural formula (21) according to claim 8 characterized by characterized by the solid-state carbon-13 CPMAS nuclear magnetic resonance spectrum of FIG. 6.

36. The crystalline free acid of the compound structural formula (21) according to claim 8 characterized by a solid-state fluorine-19 MAS nuclear magnetic resonance spectrum showing signals at −119.4, and −108.9 p.p.m.

37. The crystalline free acid of the compound structural formula (21) according to claim 8 characterized by the solid-state fluorine-19 MAS nuclear magnetic resonance spectrum of FIG. 7.

38. The crystalline free acid of the compound structural formula (21) according to claim 8 characterized by melting onset at 258° C.

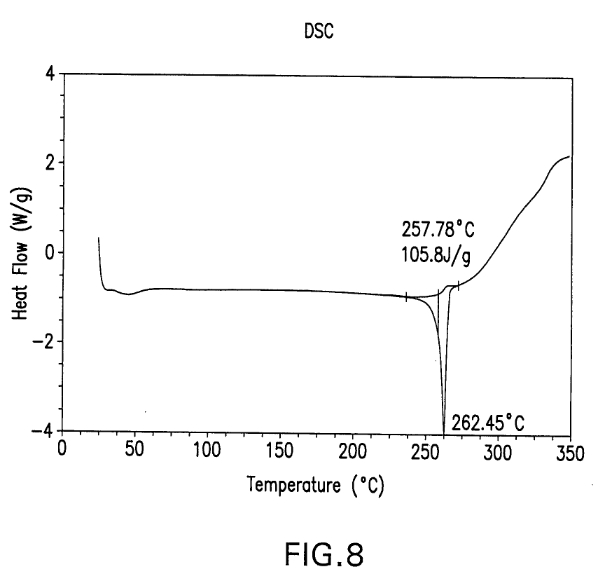

39. The crystalline free acid of the compound structural formula (21) according to claim 8 characterized by the DSC curve of FIG. 8.
